(19) United States
(12) Patent Application Publication
(10) Pub. No.: US 2003/0008327 A1
(43) Pub. Date: Jan. 9, 2003

(54) Methods and systems for identifying kinases, phosphatases, and substrates thereof

(76) Inventor: Olga Ornatskaia, Richmond Hill (CA)

Correspondence Address: ROPES & GRAY, ONE INTERNATIONAL PLACE, BOSTON, MA, 02110-2624, US

(21) Appl. No.: 10/180,224

(22) Filed: Jun. 26, 2002

Related U.S. Application Data
(60) Provisional application No. 60/300,986, filed on Jun. 26, 2001.
(60) Provisional application No. 60/313,660, filed on Aug. 20, 2001.

(51) Int. Cl. G01N 33/53; G06F 19/00; G01N 33/48; G01N 33/50

(52) U.S. Cl. 435/7.1; 702/19

(57) ABSTRACT

The instant invention provides methods to determine the phosphorylaion status or sulfation state of a polypeptide or a cell using mass spectrometry, especially ICP-MS. The invention also provides methods for identifying a substrate for a kinase using mass spectrometry. The invention further provides business method to conduct a drug discovery business. The invention further provides methods to determine the kinase activity of a peptide (such as a kinase), or the phosphatase activity of a peptide (such as a phosphatase). The invention further provides methods for identifying an inhibitor or an agonist of the kinase activity of a kinase, or an inhibitor or an agonist of the phosphatase activity of a phosphatase.

### REFERENCE TO RELATED APPLICATIONS

The application claims priority to U.S. Provisional Application 60/300,986, filed on Jun. 26, 2001, and U.S. Provisional Application 60/313,660, filed on Aug. 20, 2001, the entire contents of which are incorporated by reference herein.

### BACKGROUND OF THE INVENTION

As complete genomic sequences of various organisms continue to be established, there is an increasing interest in screening also for protein modifications to obtain more information than only the identity. One of the most common modifications is protein phosphorylation. It is estimated that ⅓ of all proteins present in a mammalian cell are phosphorylated and that kinases, enzymes responsible for that phosphorylation, constitute about 1-3% of the expressed genome. A phosphate group can modify serine, threonine, tyrosine, histidine, arginine, lysine, cysteine, glutamic acid and aspartic acid residues. However, the phosphorylation of hydroxyl groups at serine (90%), threonine (10%), or tyrosine (0.05%) residues are the most prevalent, and are involved among other processes in metabolism, cell division, cell growth, and cell differentiation.

The identification of phosphorylation sites on a protein is complicated by the fact that proteins are often only partially phosphorylated and that they are often present only at very low levels. Therefore techniques for identifying phosphorylation sites should preferably work in the low picomole to sub-picomole range, or even in the femtomole or attomole range.

The traditional way to localize the phosphorylation site on a given protein sample to be analyzed is by first labeling the proteins with radioactive phosphorus isotopes using hot γ-ATP followed by protease treatment of the protein and two-dimensional thin-layer chromatography (TLC) to isolate one or more spots using autoradiography. Site-directed mutagenesis or mutation experiments are performed to make the spot of interest disappear so that the site of mutation can be correlated to the site of phosphorylation. Though this approach is very sensitive, it is also very tedious. A more direct method entails elution of the peptide from the TLC plate followed by Edman sequencing. However, phospho-threonine and-serine esters are hydrolyzed under the conditions used for Edman sequencing. In the latter case, the dehydroalanine formed gives blank in the cycle so that only an indirect location of the site of phosphorylation is obtained.

Also, because endogenous ATP is present in the cells, in vivo labeling has a low efficiency. To obtain a detectable amount of labeled protein, large amounts of radioactivity are required, and additional safety requirements have to be fulfilled to reduce the danger of handling those amounts.

### SUMMARY OF THE INVENTION

One aspect of the invention relates to a method for identifying the phosphorylation state of a polypeptide, comprising: (i) determining, by mass spectroscopy, an elemental ratio of phosphorous to sulfur in a test sample of a polypeptide prepared under test conditions, and (ii) comparing the ratio of phosphorous to sulfur for the test sample with a ratio of phosphorous to sulfur for one or more reference samples of the test polypeptide, the reference samples being prepared under defined phosphorylation conditions, wherein a difference in the ratio of phosphorous to sulfur between the test and reference polypeptide samples indicates a difference in the level of phosphorylation resulting from the test conditions.

In another aspect, the invention provides a method for identifying the sulfation state of a polypeptide, comprising: (i) determining, by mass spectroscopy, an elemental ratio of phosphorous to sulfur in a test sample of a polypeptide prepared under test conditions, and (ii) comparing the ratio of phosphorous to sulfur for the test sample with a ratio of phosphorous to sulfur for one or more reference samples of the test polypeptide, the reference samples being prepared under defined sulfation conditions, wherein a difference in the ratio of phosphorous to sulfur between the test and the reference polypeptide samples indicates a difference in the level of sulfation resulting from the test conditions.

In one embodiment, the methods further comprises determining at least a portion of the sequence of a polypeptide identified by a difference in the level of phosphorylation or sulfation between the test and the reference polypeptide samples, preferably using mass spectrometry, such as tandem mass spectrometry (MS/MS).

In a preferred embodiment, the method further comprises searching one or more sequence databases for polypeptides, or the coding sequences therefor, having identical or homologous sequences to that determined for the identified polypeptide.

In general, the subject method relies on the use of mass spectroscopy to determine the elemental ratio of phosphorous to sulfur in a test sample of a polypeptide prepared under test conditions. By comparing the ratio of phosphorous to sulfur for the test sample with the ratio of phosphorous to sulfur for one or more reference samples of the test polypeptide, e.g., samples which were prepared under defined phosphorylation conditions, differences in the level of phosphorylation resulting from the test conditions can be observed. The sulfur level is presumably not changed between the test sample and the control sample(s) under the test conditions. In this regard, the subject method can be used to identify kinases and phosphatases and their substrates. For instance, in certain embodiments, the subject method can be used to identify, e.g., from a mixture of polypeptides, a substrate for a predetermined kinase or phosphatase. In other embodiments, the subject method can be used to identify, e.g., from a mixture of kinases or phosphatase, an enzyme that alters the phosphorylation state of a predetermined polypeptide.

In certain instances, the test conditions include exposing the test polypeptide to a kinase under conditions wherein phosphorylation of the test polypeptide occurs if it is a substrate of the kinase. In other embodiments, the test conditions including exposing a phosphorylated form of the test polypeptide to a phosphatase under conditions wherein dephosphorylation of the test polypeptide occurs if it is a substrate of the phosphatase.

In one embodiment, the test conditions include exposing the test polypeptide to a tyrosylprotein sulfotransferase under conditions wherein sulfation of the test polypeptide occurs if it is a substrate of the sulfotransferase.

In another embodiment, the method is carried out on a library of different test polypeptides.

The source of polypeptide and/or enzyme can be a whole cell in which the test polypeptide is expressed, a lysate of such a whole cell, a tissue sample, or a reconstituted or purified protein preparation/composition. For instance, where the source is a whole cell or cell lysate or tissue sample (such as those obtained from biopsy), the subject method can be used to identify kinase or phosphatase substrates whose phosphorylation status changes between two different cellular states, e.g., by comparing proteins from normal and diseased cells, differentiated and undifferentiated cells, resting and activating cells, and/or induced and uninduced cells. Where the test polypeptide(s) are recombinantly produced, the polypeptide can be a fusion protein, e.g., including a heterologous amino acid sequence for purifying the fusion protein (an affinity tag) or for immobilizing the fusion protein on a solid support such as a microtitre plate.

In a preferred embodiment, the source of polypeptide, such as tissue sample whole cell is provided in small amount, such as about the range of 10 mg, 1 mg, 0.1 mg or lower.

In certain embodiments wherein the test polypeptide is present in a mixture of polypeptides, e.g., other potential substrates or enzymes, the polypeptide is separated (e.g., prior to MS analysis) from other polypeptides on the basis of size, solubility, electric charge and/or ligand specificity. For instance, the separation can be accomplished using one or more procedures selected from the group of liquid chromatography, gel-filtration, isoelectric precipitation, electrophoresis, isoelectric focusing, ion exchange chromatography, and affinity chromatography. In certain embodiments, the polypeptides are separated using high performance liquid chromatography. In certain embodiments, the test polypeptide is separated from other polypeptides present in the test conditions on the basis of size, solubility, electric charge, and/or ligand specificity.

In certain preferred embodiments, such as where the identity of the substrate is not already known, the subject method includes a further step of determining at least a portion of the sequence of a polypeptide which is identified by differences in the level of phosphorylation or sulfation relative the to the reference polypeptide samples. In addition, it is specifically contemplated that one can search one or more protein or nucleic acid sequence databases for polypeptides, or the coding sequences therefor, having the same or similar sequences to that determined for a substrate polypeptide.

In certain preferred embodiments, the mass spectroscopy step uses inductively coupled plasma mass spectrometry (ICP-MS). In certain embodiments, the subject method detects elemental phosphorous and sulfur using laser ablation ICP-MS.

In those embodiments in which the sequence of a test polypeptide is also determined, such determinations can be made from spectra obtained using a mass spectrometer in which ionization of the sample protein is accomplished by matrix-assisted laser desorption (MALDI) ionization, electrospray (ESI), or electron impact (EI).

Another aspect of the invention provides a method for identifying a substrate for a kinase, comprising: (i) contacting a test sample of a polypeptide with a kinase under conditions wherein phosphorylation of the test polypeptide occurs if it is a substrate of the kinase, (ii) determining, by mass spectroscopy, an elemental ratio of phosphorous to sulfur in the test sample, and (iii) comparing the ratio of phosphorous to sulfur for the test sample with a ratio of phosphorous to sulfur for a reference sample of the test polypeptide not treated with the kinase, wherein an increase in the ratio of phosphorous to sulfur between the test and reference samples indicates that the test polypeptide is a substrate for the kinase.

Another aspect of the invention provides a method for identifying a substrate for a phosphatase, comprising: (i) contacting a phosphorylated sample of a test polypeptide with a phosphatase under conditions wherein dephosphorylation of the test polypeptide occurs if it is a substrate of the phosphatase, (ii) determining, by mass spectroscopy, an elemental ratio of phosphorous to sulfur in the test sample, and (iii) comparing the ratio of phosphorous to sulfur for the phosphorylated sample with a ratio of phosphorous to sulfur for a reference sample of the test polypeptide not treated with the phosphatase, wherein a decrease in the ratio of phosphorous to sulfur between the test sample and reference sample indicates that the phosphorylated test polypeptide is a substrate for the phosphatase.

Another aspect of the present invention provides a mass spectrometry system including a module that identifies the phosphorylation state of a test peptide, which module determines a level of elemental phosphorous and a level of elemental sulfur in a test sample of a polypeptide, and calculates an elemental ratio of phosphorous to sulfur for the test sample.

Yet another aspect of the present invention relates to a method of conducting a drug discovery business, comprising: (i) by the method of any of claims 1-19, identifying a kinase or phosphatase and substrate thereof; (ii) identifying agents by their ability to alter a level of phosphorylation of the substrate; (iii) conducting therapeutic profiling of agents identified in step (ii), or further analogs thereof, for efficacy and toxicity in animals; and (iv) formulating a pharmaceutical preparation including one or more agents identified in step (iii) as having an acceptable therapeutic profile.

Utilizing the methods described above, the identity of a kinase or phosphatase and/or substrate thereof are determined. Where the activity of the enzyme or the phosphorylation status of the substrate are of therapeutic relevance, agents are identified by their ability to alter the level of phosphorylation of the substrate or inhibit or activate the kinase or phosphatase. For suitable lead compounds that are identified, further therapeutic profiling of the compound, or further analogs thereof, can be carried out for assessing efficacy and toxicity in animals. Those compounds having therapeutic profiles after animal testing can be formulated into pharmaceutical preparations for use in humans or for veterinary uses. The subject business method can include an additional step of establishing a distribution system for distributing the pharmaceutical preparation for sale, and may optionally include establishing a sales group for marketing the pharmaceutical preparation.

Another aspect of the invention provides a method of conducting a drug discovery business, comprising: (i) by the method of any of claims 1-19, identifying substrate proteins which are phosphorylated or dephosphorylated as compared between two different states of a cell; (ii) identifying agents by their ability to alter a level of phosphorylation of the substrate protein(s); (iii) conducting therapeutic profiling of agents identified in step (ii), or further analogs thereof, for efficacy and toxicity in animals; and (iv) formulating a pharmaceutical preparation including one or more agents identified in step (iii) as having an acceptable therapeutic profile.

In one embodiment, the two different states compared are normal and diseased states, or differentiated and undifferentiated, or resting and activating, or induced and uninduced.

In another embodiment, the method further includes an additional step of establishing a distribution system for distributing the pharmaceutical preparation for sale, and, optionally, establishing a sales group for marketing the pharmaceutical preparation.

Another aspect of the invention provides a method of conducting a proteomics business, comprising: (i) by the method of any of claims 1-19, identifying a kinase or phosphatase and substrate thereof; (ii) licensing, to a third party, rights for further drug development of agents that alter a level of phosphorylation of the substrate.

Utilizing the methods described above, the identity of a kinase or phosphatase and/or substrate thereof are determined. Where the activity of the enzyme or the phosphorylation status of the substrate are of therapeutic relevance, the rights for further drug development of agents that alter the level of phosphorylation of the substrate, or inhibit or activate the kinase or phosphatase, are licensed to a third party.

Another aspect of the invention provides a method for determining the phosphorylation state of a cell, comprising: (i) determining, by mass spectroscopy, an elemental ratio of phosphorous to sulfur in a test sample of polypeptides prepared from one or more cells of a first phenotype, and (ii) comparing the ratio of phosphorous to sulfur for the test sample with a ratio of phosphorous to sulfur for one or more reference samples of the polypeptides, the reference samples being prepared from one or more cells of a second phenotype, wherein a difference in the ratio of phosphorous to sulfur between the test sample and the reference sample indicates a difference in a level of phosphorylation between the first and second phenotypes.

For example, the elemental ratio of phosphorous to sulfur for a test sample of polypeptides prepared from one or more cells of a first phenotype is determined by mass spectroscopy. The ratio of phosphorous to sulfur for the test sample is then compared with the ratio of phosphorous to sulfur for one or more reference samples of the polypeptides prepared from one or more cells of second phenotype. A difference in the ratio of phosphorous to sulfur between the test and reference polypeptide samples indicates a difference in the level of phosphorylation state between the first and second phenotypes.

Still another aspect of the present invention provides a method for determining the kinase activity of a kinase, comprising: (i) contacting a test sample of a polypeptide with a kinase under conditions wherein phosphorylation of the test polypeptide occurs, (ii) determining, by mass spectroscopy, a first elemental ratio of phosphorous to sulfur in the test sample at a first time, and (iii) determining, by mass spectroscopy, a second elemental ratio of phosphorous to sulfur in the test sample at a second time, whereby a difference between the first elemental ratio and the second elemental ratio and a difference between the first time and the second time are indicative of a rate constant for the kinase.

Still another aspect of the present invention provides a method for determining the phosphatase activity of a phosphatase, comprising: (i) contacting a test sample of a phosphorylated polypeptide with a phosphatase under conditions wherein dephosphorylation of the polypeptide occurs, (ii) determining, by mass spectroscopy, a first elemental ratio of phosphorous to sulfur in the test sample at a first time, and (iii) determining, by mass spectroscopy, a second elemental ratio of phosphorous to sulfur in the test sample at a second time, whereby a difference between the first elemental ratio and the second elemental ratio and a difference between the first time and the second time are indicative of a rate constant for the phosphatase.

Still another aspect of the present invention provides a method for identifying the kinase activity of a polypeptide, comprising: (i) contacting a test sample of a substrate with a test polypeptide under conditions wherein phosphorylation of the substrate occurs if the polypeptide has a kinase activity for the substrate, (ii) determining, by mass spectroscopy, an elemental ratio of phosphorous to sulfur in the test sample, and (iii) comparing the ratio of phosphorous to sulfur for the test sample with a ratio of phosphorous to sulfur for a reference sample of the substrate not treated with the test polypeptide, wherein an increase in the ratio of phosphorous to sulfur between the test sample and the reference sample indicates that the test polypeptide has a kinase activity.

Still another aspect of the present invention provides a method for identifying the phosphatase activity of a polypeptide, comprising: (i) contacting a test sample of a phosphorylated substrate with a test polypeptide under conditions wherein dephosphorylation of the substrate occurs if the polypeptide has a phosphatase activity for the substrate, (ii) determining, by mass spectroscopy, an elemental ratio of phosphorous to sulfur in the test sample, and (iii) comparing the ratio of phosphorous to sulfur for the test sample with a ratio of phosphorous to sulfur for a reference sample of the substrate not treated with the phosphatase, wherein a decrease in the ratio of phosphorous to sulfur between the test sample and reference sample indicates a phosphatase activity for the test polypeptide.

In one embodiment, the test polypeptide is a variant of a polypeptide that has a phosphatase or kinase activity for the substrate.

In another embodiment, the variant is a mutated or truncated variant of a polypeptide that has a phosphatase or kinase activity for the substrate.

Still another aspect of the present invention provides a method for identifying an inhibitor of the kinase activity of a kinase, comprising: (i) contacting a test sample of a polypeptide with a kinase and a test compound under conditions wherein phosphorylation of the polypeptide occurs in the absence of the test compound, (ii) determining, by mass spectroscopy, an elemental ratio of phosphorous to sulfur in the sample, and (iii) comparing the ratio of phosphorous to sulfur for the test sample with a ratio of phosphorous to sulfur for a reference sample of the polypeptide treated with the kinase in the absence of the test compound, wherein a decreased ratio of phosphorous to sulfur in the test sample as compared to the reference sample indicates that the test compound inhibits the kinase activity.

Still another aspect of the present invention provides a method for identifying an inhibitor of the phosphatase activity of a phosphatase, comprising: (i) contacting a test sample of a phosphorylated polypeptide with a phosphatase and a test compound under conditions wherein dephosphorylation of test polypeptide occurs in the absence of the test compound, (ii) determining, by mass spectroscopy, an elemental ratio of phosphorous to sulfur in the test sample, and (iii) comparing the ratio of phosphorous to sulfur for the test sample with a ratio of phosphorous to sulfur for a reference sample of the substrate treated with the phosphatase in the absence of the test compound; wherein an increased ratio of phosphorous to sulfur in the test sample as compared to the reference sample indicates inhibition of the phosphatase activity by the test compound.

Still another aspect of the present invention provides a method for identifying an agonist of the kinase activity of a kinase, comprising: (i) contacting a test sample of a polypeptide with a kinase and a test compound under conditions wherein phosphorylation of the polypeptide occurs in the absence of the test compound, (ii) determining, by mass spectroscopy, an elemental ratio of phosphorous to sulfur in the sample, and (iii) comparing the ratio of phosphorous to sulfur for the test sample with a ratio of phosphorous to sulfur for a reference sample of the polypeptide treated with the kinase in the absence of the test compound, wherein an increased ratio of phosphorous to sulfur in the test sample as compared to the reference sample indicates that the test compound agonizes the kinase activity.

Still another aspect of the present invention provides a method for identifying an agonist of the phosphatase activity of a phosphatase, comprising: (i) contacting a test sample of a phosphorylated polypeptide with a phosphatase and a test compound under conditions wherein dephosphorylation of test polypeptide occurs in the absence of the test compound, (ii) determining, by mass spectroscopy, an elemental ratio of phosphorous to sulfur in the test sample, and (iii) comparing the ratio of phosphorous to sulfur for the test sample with a ratio of phosphorous to sulfur for a reference sample of the substrate treated with the phosphatase in the absence of the test compound, wherein a decreased ratio of phosphorous to sulfur in the test sample as compared to the reference sample indicates that the test compound agonizes the phosphatase activity.

Still another aspect of the present invention provides a method for identifying the sulfation state of a polypeptide. As above, the elemental ratio of phosphorous to sulfur in a test sample is determined by mass spectroscopy and compared to one or more reference samples. In certain embodiments, the test sample has been contacted with a tyrosylprotein sulfotransferase under conditions wherein sulfation of the test polypeptide occurs if it is a substrate of the sulfotransferase.

In certain embodiments, the invention provides a high-throughput method for determining the gross phosphorylation state of a polypeptide sample. In certain embodiments, the polypeptide sample can be a processed or unprocessed sample of lymph, blood, serum, urine, saliva, or another biological fluid from a patient, or proteins obtained from such a fluid.

### BRIEF DESCRIPTION OF THE DRAWINGS

FIG. 1 Autophosphorylation kinase assay determined by P and S content.

FIG. 2 Kinase substrate phosphorylation determined by P and S content.

FIG. 3 Results for PO/SO ratio (P and S content ratio) difference between human normal colorectal epithelium and human colorectal carcinoma sample. Both samples were obtained from the same patient. Amount of material used is extremely low—about 1 mg, and only 1% was used for the ICP-MS analysis. Thus, very small amount of biopsy material can be used to distinguish normal from malignant tissue.

### DETAILED DESCRIPTION OF THE INVENTION

I. Overview

The present invention provides a method for the determination of kinase or phosphatase activity of protein samples. Certain embodiments of the subject method are particularly well suited for high-throughput analysis of samples, such as may be provided in multiwell-plate format, e.g., microtitre plates, or arrayed on solid supports. The method is based on the determination of the phosphorylated state of the sample proteins by measuring the elemental ratio of phosphorous to sulfur (P/S). This ratio can be determined using, e.g., inductively coupled plasma mass spectrometry (ICP-MS). The samples can be naturally occurring (native) proteins or recombinant proteins. Further to the invention's ability to measure the kinase activity of the samples, it can be readily adopted for other kinase-related functions such as measurement of autophosphorylation or phosphatase activity.

The subject methods can be further extended for the purpose of evaluating small-molecule inhibition (or activation) of the kinase or phosphatase activity of protein samples.

II. Definitions

“Inductively Coupled Plasma Mass Spectrometry” or “ICP-MS” refers to a multi-element technique that uses a plasma source to dissociate the sample into its constituent atoms or ions. In this case, it is the ions themselves that are-detected. The ions are extracted from the central channel of the plasma and pass into the mass spectrometer, where they are separated based on their atomic mass-to-charge ratio by a quadrupole or magnetic sector analyzer.

The high number of ions produced, combined with very low backgrounds, provides the best detection limits available for most elements, normally in the parts-per-trillion range. However, it is important to remember that detection limits can be no better than lab cleanliness allows; to realize its full potential, an ICP-MS requires a clean room environment.

“Homology” or “identity” or “similarity” refers to sequence similarity between two peptides or between two nucleic acid molecules. Homology and identity can each be determined by comparing a position in each sequence that may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present invention.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “genomic information” includes protein coding regions, introns and other non-coding sequences, and other such structures that commonly appear genomic sequences. It is also meant to include the reading frame for proteins as encoded by a gene.

“ORF” or “Open Reading Frame” is a nucleotide sequence that can be translated into a polypeptide. Such a stretch of sequence is uninterrupted by a stop codon. An ORF that represents the coding sequence for a full protein begins with an ATG “start” codon and terminates with one of the three “stop” codons. For the purposes of this application, an ORF may be any part of a coding sequence, with or without start and/or stop codons. “ORF” and “CDS” may be used interchangeably.

The term “annotation” refers to the description of an ORF, introns and other genomic features.

“Abnormality” or “abnormal” refers to a level that is statistically different from the level observed in organisms not suffering from a disease or condition. It may be characterized by an excess amount, intensity or duration of signal, or a deficient amount, intensity or duration of a protein in general or a particular form of a protein. An abnormality may be realized in a cell as an abnormality in cell function, viability, or differentiation state. An abnormal interaction level may be greater or less than a normal level and may impair the performance or function of an organism.

The terms “compound”, “test compound” and “molecule” are used herein interchangeably and are meant to include, but are not limited to, peptides, nucleic acids, carbohydrates, small organic molecules, natural product extract libraries, and any other molecules (including, but not limited to, chemicals, metals and organometallic compounds).

The term “agonist” as used herein, refers to a molecule that augments a particular activity, such as kinase-mediated phosphorylation or phosphatase-mediated dephosphorylation. The stimulation may be direct, or indirect, or by a competitive or non-competitive mechanism. The term “antagonist”, as used herein, refers to a molecule that decreases the amount of or duration of a particular activity, such as kinase-mediated phosphorylation or phosphatase-mediated dephosphorylation. The inhibition may be direct, or indirect, or by a competitive or non-competitive mechanism. Agonists and antagonists may include proteins, including antibodies, that compete for binding at a binding region of a member of the complex, nucleic acids including anti-sense molecules, carbohydrates, or any other molecules, including, for example, chemicals, metals, organometallic agents, etc.

As used herein the term “animal” refers to mammals, preferably mammals such as humans.

A “chimeric protein” or “fusion protein” is a fusion of a first amino acid sequence encoding a polypeptide with a second amino acid sequence defining a domain foreign to and not substantially homologous with any domain of the protein. A chimeric protein may present a foreign domain that is found (albeit in a different protein) in an organism that also expresses the first protein, or it may be an “interspecies”, “intergenic”, etc., fusion of protein structures expressed by different kinds of organisms.

The term “isolated”, as used herein with reference to the subject proteins, refers to a preparation of protein or protein complex that is essentially free from contaminating proteins that normally would be present in association with the protein or complex, e.g., in the cellular milieu in which the protein or complex is found endogenously.

As used herein, “phenotype” refers to the entire physical, biochemical, and physiological makeup of a cell, e.g., having any one trait or any group of traits.

The term “recombinant protein” refers to a protein of the present invention which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

By “semi-purified”, with respect to protein preparations, it is meant that the proteins have been previously separated from other cellular or viral proteins. For instance, in contrast to whole cell lysates, the proteins of reconstituted conjugation system, together with the substrate protein, can be present in the mixture to at least 50% purity relative to all other proteins in the mixture, more preferably are present at least 75% purity, and even more preferably are present at 90-95% purity.

The term “semi-purified cell extract” or, alternatively, “fractionated lysate”, as used herein, refers to a cell lysate which has been treated so as to substantially remove at least one component of the whole cell lysate, or to substantially enrich at least one component of the whole cell lysate. “Substantially remove”, as used herein, means to remove at least 10%, more preferably at least 50%, and still more preferably at least 80%, of the component of the whole cell lysate. “Substantially enrich”, as used herein, means to enrich by at least 10%, more preferably by at least 30%, and still more preferably at least about 50%, at least one component of the whole cell lysate compared to another component of the whole cell lysate.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the invention.

III. Mass Spectrometers and Detection Methods

Mass Spectrometry

Mass spectrometry, also called mass spectroscopy, is an instrumental approach that allows for the gas phase generation of ions as well as their separation and detection. The five basic parts of any mass spectrometer include: a vacuum system; a sample introduction device; an ionization source; a mass analyzer; and an ion detector. A mass spectrometer determines the molecular weight of chemical compounds by ionizing, separating, and measuring molecular ions according to their mass-to-charge ratio (m/z). The ions are generated in the ionization source by inducing either the loss or the gain of a charge (e.g. electron ejection, protonation, or deprotonation). Once the ions are formed in the gas phase they can be electrostatically directed into a mass analyzer, separated according to mass and finally detected. The result of ionization, ion separation, and detection is a mass spectrum that can provide molecular weight or even structural information.

A common requirement of all mass spectrometers is a vacuum. A vacuum is necessary to permit ions to reach the detector without colliding with other gaseous molecules. Such collisions would reduce the resolution and sensitivity of the instrument by increasing the kinetic energy distribution of the ion's inducing fragmentation, or preventing the ions from reaching the detector. In general, maintaining a high vacuum is crucial to obtaining high quality spectra.

The sample inlet is the interface between the sample and the mass spectrometer. One approach to introducing sample is by placing a sample on a probe which is then inserted, usually through a vacuum lock, into the ionization region of the mass spectrometer. The sample can then be heated to facilitate thermal desorption or undergo any number of high-energy desorption processes used to achieve vaporization and ionization.

Capillary infusion is often used in sample introduction because it can efficiently introduce small quantities of a sample into a mass spectrometer without destroying the vacuum. Capillary columns are routinely used to interface the ionization source of a mass spectrometer with other separation techniques including gas chromatography (GC) and liquid chromatography (LC). Gas chromatography and liquid chromatography can serve to separate a solution into its different components prior to mass analysis. Prior to the 1980's, interfacing liquid chromatography with the available ionization techniques was unsuitable because of the low sample concentrations and relatively high flow rates of liquid chromatography. However, new ionization techniques such as electrospray were developed that now allow LC/MS to be routinely performed. One variation of the technique is that high performance liquid chromatography (HPLC) can now be directly coupled to mass spectrometer for integrated sample separation/preparation and mass spectrometer analysis.

In terms of sample ionization, two of the most recent techniques developed in the mid 1980's have had a significant impact on the capabilities of Mass Spectrometry: Electrospray Ionization (ESI) and Matrix Assisted Laser Desorption/Ionization (MALDI). ESI is the production of highly charged droplets which are treated with dry gas or heat to facilitate evaporation leaving the ions in the gas phase. MALDI uses a laser to desorb sample molecules from a solid or liquid matrix containing a highly UV-absorbing substance.

The MALDI-MS technique is based on the discovery in the late 1980s that an analyte consisting of, for example, large nonvolatile molecules such as proteins, embedded in a solid or crystalline “matrix” of laser light-absorbing molecules can be desorbed by laser irradiation and ionized from the solid phase into the gaseous or vapor phase, and accelerated as intact molecular ions towards a detector of a mass spectrometer. The “matrix” is typically a small organic acid mixed in solution with the analyte in a 10,000:1 molar ratio of matrix/analyte. The matrix solution can be adjusted to neutral pH before mixing with the analyte.

The MALDI ionization surface may be composed of an inert material or else modified to actively capture an analyte. For example, an analyte binding partner may be bound to the surface to selectively absorb a target analyte or the surface may be coated with a thin nitrocellulose film for nonselective binding to the analyte. The surface may also be used as a reaction zone upon which the analyte is chemically modified, e.g., CNBr degradation of protein. See Bai et al, Anal. Chem. 67, 1705-1710 (1995).

Metals such as gold, copper and stainless steel are typically used to form MALDI ionization surfaces. However, other commercially-available inert materials (e.g., glass, silica, nylon and other synthetic polymers, agarose and other carbohydrate polymers, and plastics) can be used where it is desired to use the surface as a capture region or reaction zone. The use of Nation and nitrocellulose-coated MALDI probes for on-probe purification of PCR-amplified gene sequences is described by Liu et al., Rapid Commun. Mass Spec. 9:735-743 (1995). Tang et al. have reported the attachment of purified oligonucleotides to beads, the tethering of beads to a probe element, and the use of this technique to capture a complimentary DNA sequence for analysis by MALDI-TOF MS (reported by K. Tang et al., at the May 1995 TOF-MS workshop, R. J. Cotter (Chairperson); K. Tang et al., Nucleic Acids Res. 23, 3126-3131, 1995). Alternatively, the MALDI surface may be electrically—or magnetically activated to capture charged analytes and analytes anchored to magnetic beads respectively.

Aside from MALDI, Electrospray Ionization Mass Spectrometry (ESI/MS) has been recognized as a significant tool used in the study of proteins, protein complexes and biomolecules in general. ESI is a method of sample introduction for mass spectrometric analysis whereby ions are formed at atmospheric pressure and then introduced into a mass spectrometer using a special interface. Large organic molecules, of molecular weight over 10,000 Daltons, may be analyzed in a quadrupole mass spectrometer using ESI.

In ESI, a sample solution containing molecules of interest and a solvent is pumped into an electrospray chamber through a fine needle. An electrical potential of several kilovolts may be applied to the needle for generating a fine spray of charged droplets. The droplets may be sprayed at atmospheric pressure into a chamber containing a heated gas to vaporize the solvent. Alternatively, the needle may extend into an evacuated chamber, and the sprayed droplets are then heated in the evacuated chamber. The fine spray of highly charged droplets releases molecular ions as the droplets vaporize at atmospheric pressure. In either case, ions are focused into a beam, which is accelerated by an electric field, and then analyzed in a mass spectrometer.

Because electrospray ionization occurs directly from solution at atmospheric pressure, the ions formed in this process tend to be strongly solvated. To carry out meaningful mass measurements, solvent molecules attached to the ions should be efficiently removed, that is, the molecules of interest should be “desolvated.” Desolvation can, for example, be achieved by interacting the droplets and solvated ions with a strong countercurrent flow (6-9 l/m) of a heated gas before the ions enter into the vacuum of the mass analyzer.

Other well-known ionization methods may also be used. For example, electron ionization (also known as electron bombardment and electron impact), atmospheric pressure chemical ionization (APCI), fast atom Bombardment (FAB), or chemical ionization (CI).

Immediately following ionization, gas phase ions enter a region of the mass spectrometer known as the mass analyzer. The mass analyzer is used to separate ions within a selected range of mass to charge ratios. This is an important part of the instrument because it plays a large role in the instrument's accuracy and mass range. Ions are typically separated by magnetic fields, electric fields, and/or measurement of the time an ion takes to travel a fixed distance.

If all ions with the same charge enter a magnetic field with identical kinetic energies a definite velocity will be associated with each mass and the radius will depend on the mass. Thus a magnetic field can be used to separate a monoenergetic ion beam into its various mass components. Magnetic fields will also cause ions to form fragment ions. If there is no kinetic energy of separation of the fragments the two fragments will continue along the direction of motion with unchanged velocity. Generally, some kinetic energy is lost during the fragmentation process creating noninteger mass peak signals which can be easily identified. Thus, the action of the magnetic field on fragmented ions can be used to give information on the individual fragmentation processes taking place in the mass spectrometer.

Electrostatic fields exert radial forces on ions attracting them towards a common center. The radius of an ion's trajectory will be proportional to the ion's kinetic energy as it travels through the electrostatic field. Thus an electric field can be used to separate ions by selecting for ions that travel within a specific range of radii which is based on the kinetic energy and is also proportion to the mass of each ion.

Quadrupole mass analyzers have been used in conjunction with electron ionization sources since the 1950s. Quadrupoles are four precisely parallel rods with a direct current (DC) voltage and a superimposed radio-frequency (RF) potential. The field on the quadrupoles determines which ions are allowed to reach the detector. The quadrupoles thus function as a mass filter. As the field is imposed, ions moving into this field region will oscillate depending on their mass-to-charge ratio and, depending on the radio frequency field, only ions of a particular m/z can pass through the filter. The m/z of an ion is therefore determined by correlating the field applied to the quadrupoles with the ion reaching the detector. A mass spectrum can be obtained by scanning the RF field. Only ions of a particular m/z are allowed to pass through.

Electron ionization coupled with quadrupole mass analyzers can be employed in practicing the instant invention. Quadrupole mass analyzers have found new utility in their capacity to interface with electrospray ionization. This interface has three primary advantages. First, quadrupoles are tolerant of relatively poor vacuums (˜5×10⁻⁵ torr), which makes it well-suited to electrospray ionization since the ions are produced under atmospheric pressure conditions. Secondly, quadrupoles are now capable of routinely analyzing up to an m/z of 3000, which is useful because electrospray ionization of proteins and other biomolecules commonly produces a charge distribution below m/z 3000. Finally, the relatively low cost of quadrupole mass spectrometers makes them attractive as electrospray analyzers.

The ion trap mass analyzer was conceived of at the same time as the quadrupole mass analyzer. The physics behind both of these analyzers is very similar. In an ion trap the ions are trapped in a radio frequency quadrupole field. One method of using an ion trap for mass spectrometry is to generate ions externally with ESI or MALDI, using ion optics for sample injection into the trapping volume. The quadrupole ion trap typically consist of a ring electrode and two hyperbolic endcap electrodes. The motion of the ions trapped by the electric field resulting from the application of RF and DC voltages allows ions to be trapped or ejected from the ion trap. In the normal mode the RF is scanned to higher voltages, the trapped ions with the lowest m/z and are ejected through small holes in the endcap to a detector (a mass spectrum is obtained by resonantly exciting the ions and thereby ejecting from the trap and detecting them). As the RF is scanned further, higher m/z ratios become are ejected and detected. It is also possible to isolate one ion species by ejecting all others from the trap. The isolated ions can subsequently be fragmented by collisional activation and the fragments detected. The primary advantages of quadrupole ion traps is that multiple collision-induced dissociation experiments can be performed without having multiple analyzers. Other important advantages include its compact size, and the ability to trap and accumulate ions to increase the signal-to-noise ratio of a measurement.

Quadrupole ion traps can be used in conjunction with electrospray ionization MS/MS experiments in the instant invention.

The earliest mass analyzers separated ions with a magnetic field. In magnetic analysis, the ions are accelerated (using an electric field) and are passed into a magnetic field. A charged particle traveling at high speed passing through a magnetic field will experience a force, and travel in a circular motion with a radius depending upon the m/z and speed of the ion. A magnetic analyzer separates ions according to their radii of curvature, and therefore only ions of a given m/z will be able to reach a point detector at any given magnetic field. A primary limitation of typical magnetic analyzers is their relatively low resolution.

In order to improve resolution, single-sector magnetic instruments have been replaced with double-sector instruments by combining the magnetic mass analyzer with an electrostatic analyzer. The electric sector acts as a kinetic energy filter allowing only ions of a particular kinetic energy to pass through its field, irrespective of their mass-to-charge ratio. Given a radius of curvature, R, and a field, E, applied between two curved plates, the equation R=2V/E allows one to determine that only ions of energy V will be allowed to pass. Thus, the addition of an electric sector allows only ions of uniform kinetic energy to reach the detector, thereby increasing the resolution of the two sector instrument to 100,000. Magnetic double-focusing instrumentation is commonly used with FAB and EI ionization, however they are not widely used for electrospray and MALDI ionization sources primarily because of the much higher cost of these instruments. But in theory, they can be employed to practice the instant invention.

ESI and MALDI-MS commonly use quadrupole and time-of-flight mass analyzers, respectively. The limited resolution offered by time-of-flight mass analyzers, combined with adduct formation observed with MALDI-MS, results in accuracy on the order of 0.1% to a high of 0.01%, while ESI typically has an accuracy on the order of 0.01%. Both ESI and MALDI are now being coupled to higher resolution mass analyzers such as the ultrahigh resolution (>10⁵) mass analyzer. The result of increasing the resolving power of ESI and MALDI mass spectrometers is an increase in accuracy for biopolymer analysis.

Fourier-transform ion cyclotron resonance (FTMS) offers two distinct advantages, high resolution and the ability to tandem mass spectrometry experiments. FTMS is based on the principle of a charged particle orbiting the presence of a magnetic field. While the ions are orbiting, a radio frequency (RF) signal is used to excite them and as a result of this RF excitation, the ions produce a detectable image current. The time-dependent image current can then be Fourier transformed to obtain the component frequencies of the different ions which correspond to their m/z.

Coupled to ESI and MALDI, FTMS offers high accuracy with errors as low as ±0.001%. The ability to distinguish individual isotopes of a protein of mass 29,000 is demonstrated.

A time-of-flight (TOF) analyzer is one of the simplest mass analyzing devices and is commonly used with MALDI ionization. Time-of-flight analysis is based on accelerating a set of ions to a detector with the same amount of energy. Because the ions have the same energy, yet a different mass, the ions reach the detector at different times. The smaller ions reach the detector first because of their greater velocity and the larger ions take longer, thus the analyzer is called time-of-flight because the mass is determine from the ions' time of arrival.

The arrival time of an ion at the detector is dependent upon the mass, charge, and kinetic energy of the ion. Since kinetic energy (KE) is equal to ½ mv² or velocity v=(2KE/m)^{½}, ions will travel a given distance, d, within a time, t, where t is dependent upon their m/z.

The magnetic double-focusing mass analyzer has two distinct parts, a magnetic sector and an electrostatic sector. The magnet serves to separate ions according to their mass-to-charge ratio since a moving charge passing through a magnetic field will experience a force, and travel in a circular motion with a radius of curvature depending upon the m/z of the ion. A magnetic analyzer separates ions according to their radii of curvature, and therefore only ions of a given m/z will be able to reach a point detector at any given magnetic field. A primary limitation of typical magnetic analyzers is their relatively low resolution. The electric sector acts as a kinetic energy filter allowing only ions of a particular kinetic energy to pass through its field, irrespective of their mass-to-charge ratio. Given a radius of curvature, R, and a field, E, applied between two curved plates, the equation R=2V/E allows one to determine that only ions of energy V will be allowed to pass. Thus, the addition of an electric sector allows only ions of uniform kinetic energy to reach the detector, thereby increasing the resolution of the two sector instrument.

The new ionization techniques are relatively gentle and do not produce a significant amount of fragment ions, this is in contrast to electron ionization (EI) which produces many fragment ions. To generate more information on the molecular ions generated in the ESI and MALDI ionization sources, it has been necessary to apply techniques such as tandem mass spectrometry (MS/MS), to induce fragmentation. Tandem mass spectrometry (abbreviated MSn—where n refers to the number of generations of fragment ions being analyzed) allows one to induce fragmentation and mass analyze the fragment ions. This is accomplished by collisionally generating fragments from a particular ion and then mass analyzing the fragment ions.

Fragmentation can be achieved by inducing ion/molecule collisions by a process known as collision-induced dissociation (CID) or also known as collision-activated dissociation (CAD). CID is accomplished by selecting an ion of interest with a mass filter/analyzer and introducing that ion into a collision cell. A collision gas (typically Ar, although other noble gases can also be used) is introduced into the collision cell, where the selected ion collides with the argon atoms, resulting in fragmentation. The fragments can then be analyzed to obtain a fragment ion spectrum. The abbreviation MSn is applied to processes which analyze beyond the initial fragment ions (MS2) to second (MS3) and third generation fragment ions (MS4). Tandem mass analysis is primarily used to obtain structural information, such as protein or polypeptide sequence, in the instant invention.

In certain instruments, such as those by JEOL USA, Inc. (Peabody, Mass.), the magnetic and electric sectors in any JEOL magnetic sector mass spectrometer can be scanned together in “linked scans” that provide powerful MS/MS capabilities without requiring additional mass analyzers. Linked scans can be used to obtain product-ion mass spectra, precursor-ion mass spectra, and constant neutral-loss mass spectra. These can provide structural information and selectivity even in the presence of chemical interferences. Constant neutral loss spectrum essentially “lifts out” only the interested peaks away from all the background peaks, hence removing the need for class separation and purification. Neutral loss spectrum can be routinely generated by a number of commercial mass spectrometer instruments (such as the one used in the Example section). JEOL mass spectrometers can also perform fast linked scans for GC/MS/MS and LC/MS/MS experiments.

Once the ion passes through the mass analyzer it is then detected by the ion detector, the final element of the mass spectrometer. The detector allows a mass spectrometer to generate a signal (current) from incident ions, by generating secondary electrons, which are further amplified. Alternatively some detectors operate by inducing a current generated by a moving charge. Among the detectors described, the electron multiplier and scintillation counter are probably the most commonly used and convert the kinetic energy of incident ions into a cascade of secondary electrons. Ion detection can typically employ Faraday Cup, Electron Multiplier, Photomultiplier Conversion Dynode (Scintillation Counting or Daly Detector), High-Energy Dynode Detector (HED), Array Detector, or Charge (or Inductive) Detector.

The introduction of computers for MS work entirely altered the manner in which mass spectrometry was performed. Once computers were interfaced with mass spectrometers it was possible to rapidly perform and save analyses. The introduction of faster processors and larger storage capacities has helped launch a new era in mass spectrometry. Automation is now possible allowing for thousands of samples to be analyzed in a single day. Te use of computer also helps to develop mass spectra databases which can be used to store experimental results. Software packages not only helped to make the mass spectrometer more user friendly but also greatly expanded the instrument's capabilities.

The ability to analyze complex mixtures has made MALDI and ESI very useful for the examination of proteolytic digests, an application otherwise known as protein mass mapping. Through the application of sequence specific proteases, protein mass mapping allows for the identification of protein primary structure. Performing mass analysis on the resulting proteolytic fragments thus yields information on fragment masses with accuracy approaching ±5 ppm, or ±0.005 Da for a 1,000 Da peptide. The protease fragmentation pattern is then compared with the patterns predicted for all proteins within a database and matches are statistically evaluated. Since the occurrence of Arg and Lys residues in proteins is statistically high, trypsin cleavage (specific for Arg and Lys) generally produces a large number of fragments which in turn offer a reasonable probability for unambiguously identifying the target protein.

The characterization of methylation status of a given polypeptide is extremely important for the study of PRMT and their functions in regulating a number of important biological cellular functions. Sometimes, the exact identity of a polypeptide being analyzed is not certain. In these situations, mass spectrometry has the added advantage of identifying polypeptide sequences containing the methylated arginine residue(s). The primary tools in these protein identification experiments are mass spectrometry, proteases, and computer-facilitated data analysis. As a result of generating intact ions, the molecular weight information on the peptides/proteins are quite unambiguous. Sequence specific enzymes can then provide protein fragments that can be associated with proteins within a database by correlating observed and predicted fragment masses. The success of this strategy, however, relies on the existence of the protein sequence within the database. With the availability of the human genome sequence (which indirectly contain the sequence information of all the proteins in the human body) and genome sequences of other organisms (mouse, rat, Drosophila, C. elegans, bacteria, yeasts, etc.), identification of the proteins can be quickly determined simply by measuring the mass of proteolytic fragments.

Protease digestion

One aspect of the instant invention is that peptide fragments ending with lysine or arginine residues can be used for sequencing with tandem mass spectrometry. While trypsin is the preferred the protease, many different enzymes can be used to perform the digestion to generate peptide fragments ending with Lys or Arg residues. For instance, in page 886 of a 1979 publication of Enzymes (Dixon, M. et al. ed., 3rd edition, Academic Press, New York and San Francisco, the content of which is incorporated herein by reference), a host of enzymes are listed which all have preferential cleavage sites of either Arg- or Lys- or both, including Trypsin [EC 3.4.21.4], Thrombin [EC 3.4.21.5], Plasmin [EC 3.4.21.7], Kallilkrein [EC 3.4.21.8], Acrosin [EC 3.4.21.10], and Coagulation factor Xa [EC 3.4.21.6]. Particularly, Acrosin is the Trypsin-like enzyme of spermatoza, and it is not inhibited by α1-antitrypsin. Plasmin is cited to have higher selectivity than Trypsin, while Thrombin is said to be even more selective. However, this list of enzymes are for illustration purpose only and is not intended to be limiting in any way. Other enzymes known to reliably and predictably perform digestions to generate the polypeptide fragments as described in the instant invention are also within the scope of the invention.

Sequence and Literature Databases and Database Search

The raw data of mass spectrometry will be compared to public, private or commercial databases to determine the identity of polypeptides.

BLAST search can be performed at the NCBI's (National Center for Biotechnology Information) BLAST website. According to the NCBI BLAST website, BLAST® (Basic Local Alignment Search Tool) is a set of similarity search programs designed to explore all of the available sequence databases regardless of whether the query is protein or DNA. The BLAST programs have been designed for speed, with a minimal sacrifice of sensitivity to distant sequence relationships. The scores assigned in a BLAST search have a well-defined statistical interpretation, making real matches easier to distinguish from random background hits. BLAST uses a heuristic algorithm which seeks local as opposed to global alignments and is therefore able to detect relationships among sequences which share only isolated regions of similarity (Altschul et al., 1990, J. Mol. Biol. 215: 403-10). The BLAST website also offer a “BLAST course,” which explains the basics of the BLAST algorithm, for a better understanding of BLAST.

For protein sequence search, several protein-protein BLAST can be used. Protein BLAST allows one to input protein sequences and compare these against other protein sequences.

“Standard protein-protein BLAST” takes protein sequences in FASTA format, GenBank Accession numbers or GI numbers and compares them against the NCBI protein databases (see below).

“PSI-BLAST” (Position Specific Iterated BLAST) uses an iterative search in which sequences found in one round of searching are used to build a score model for the next round of searching. Highly conserved positions receive high scores and weakly conserved positions receive scores near zero. The profile is used to perform a second (etc.) BLAST search and the results of each “iteration” used to refine the profile. This iterative searching strategy results in increased sensitivity.

“PHI-BLAST” (Pattern Hit Initiated BLAST) combines matching of regular expression pattern with a Position Specific iterative protein search. PHI-BLAST can locate other protein sequences which both contain the regular expression pattern and are homologous to a query protein sequence.

“Search for short, nearly exact sequences” is an option similar to the standard protein-protein BLAST with the parameters set automatically to optimize for searching with short sequences. A short query is more likely to occur by chance in the database. Therefore increasing the Expect value threshold, and also lowering the word size is often necessary before results can be returned. Low Complexity filtering has also been removed since this filters out larger percentage of a short sequence, resulting in little or no query sequence remaining. Also for short protein sequence searches the Matrix is changed to PAM-30 which is better suited to finding short regions of high similarity.

The databases that can be searched by the BLAST program is user selected, and is subject to frequent updates at NCBI. The most commonly used ones are:

Nr: All non-redundant GenBank CDS translations+PDB+SwissProt+PIR+PRF;

Month: All new or revised GenBank CDS translation+PDB+SwissProt+PIR+PRF released in the last 30 days;

Swissprot: Last major release of the SWISS-PROT protein sequence database (no updates);

Drosophila genome: Drosophila genome proteins provided by Celera and Berkeley Drosophila Genome Project (BDGP);

S. cerevisiae: Yeast (Saccharomyces cerevisiae) genomic CDS translations;

E. coli: Escherichia coli genomic CDS translations;

Pdb: Sequences derived from the 3-dimensional structure from Brookhaven Protein Data Bank;

Alu: Translations of select Alu repeats from REPBASE, suitable for masking Alu repeats from query sequences. It is available by anonymous FTP from the NCBI website. See “Alu alert” by Claverie and Makalowski, Nature vol. 371, page 752 (1994).

Some of the BLAST databases, like SwissProt, PDB and Kabat are complied outside of NCBI. Other like ecoli, dbEST and month, are subsets of the NCBI databases. Other “virtual Databases” can be created using the “Limit by Entrez Query” option.

The Welcome Trust Sanger Institute offer the Ensembl sofeware system which produces and maintains automatic annotation on eukaryotic genomes. All data and codes can be downloaded without constraints from the Sanger Centre website. The Centre also provides the Ensembl's International Protein Index databases which contain more than 90% of all known human protein sequences and additional prediction of about 10,000 proteins with supporting evidence. All these can be used for database search purposes.

In addition, many commercial databases are also available for search purposes. For example, Celera has sequenced the whole human genome and offers commercial access to its proprietary annotated sequence database (Discovery™ database).

Various softwares can be employed to search these databases. The probability search sofeware Mascot (Matrix Science Ltd.). Mascot utilizes the Mowse search algorithm and scores the hits using a probabilistic measure (Perkins et al., 1999, Electrophoresis 20: 3551-3567, the entire contents are incorporated herein by reference). The Mascot score is a function of the database utilized, and the score can be used to assess the null hypothesis that a particular match occurred by chance. Specifically, a Mascot score of 46 implies that the chance of a random hit is less than 5%. However, the total score consists of the individual peptide scores, and occasionally, a high total score can derive from many poor hits. To exclude this possibility, only “high quality” hits—those with a total score >46 with at least a single peptide match with a score of 30 ranking number 1—are considered.

Other similar softwares can also be used according to manufacturer's suggestion.

To determine if a particular protein is novel, that is, whether it is not previously found to localize to a particular subcellular compartment or organelle, further search of bioinformatics databases are necessary. One useful database for this type of literature search is PubMed.

PubMed, available via the NCBI Entrez retrieval system, was developed by the National Center for Biotechnology Information (NCBI) at the National Library of Medicine (NLM), located at the National Institutes of Health (NIH). The PubMed database was developed in conjunction with publishers of biomedical literature as a search tool for accessing literature citations and linking to full-text journal articles at web sites of participating publishers.

Publishers participating in PubMed electronically supply NLM with their citations prior to or at the time of publication. If the publisher has a web site that offers full-text of its journals, PubMed provides links to that site, as well as sites to other biological data, sequence centers, etc. User registration, a subscription fee, or some other type of fee may be required to access the full-text of articles in some journals.

In addition, PubMed provides a Batch Citation Matcher, which allows publishers (or other outside users) to match their citations to PubMed entries, using bibliographic information such as journal, volume, issue, page number, and year. This permits publishers easily to link from references in their published articles directly to entries in PubMed.

PubMed provides access to bibliographic information which includes MEDLINE as well as:

The out-of-scope citations (e.g., articles on plate tectonics or astrophysics) from certain MEDLINE journals, primarily general science and chemistry journals, for which the life sciences articles are indexed for MEDLINE.

Citations that precede the date that a journal was selected for MEDLINE indexing.

Some additional life science journals that submit full text to PubMed Central and receive a qualitative review by NLM.

PubMed also provides access and links to the integrated molecular biology databases included in NCBI's Entrez retrieval system. These databases contain DNA and protein sequences, 3-D protein structure data, population study data sets, and assemblies of complete genomes in an integrated system.

MEDLINE is the NLM's premier bibliographic database covering the fields of medicine, nursing, dentistry, veterinary medicine, the health care system, and the preclinical sciences. MEDLINE contains bibliographic citations and author abstracts from more than 4,300 biomedical journals published in the United States and 70 other countries. The file contains over 11 million citations dating back to the mid-1960's. Coverage is worldwide, but most records are from English-language sources or have English abstracts.

PubMed's in-process records provide basic citation information and abstracts before the citations are indexed with NLM's MeSH Terms and added to MEDLINE. New in process records are added to PubMed daily and display with the tag [PubMed—in process]. After MeSH terms, publication types, GenBank accession numbers, and other indexing data are added, the completed MEDLINE citations are added weekly to PubMed.

Citations received electronically from publishers appear in PubMed with the tag [PubMed—as supplied by publisher]. These citations are added to PubMed Tuesday through Saturday. Most of these progress to In Process, and later to MEDLINE status. Not all citations will be indexed for MEDLINE and are tagged, [PubMed—as supplied by publisher].

The Batch Citation Matcher allows users to match their own list of citations to PubMed entries, using bibliographic information such as journal, volume, issue, page number, and year. The Citation Matcher reports the corresponding PMID. This number can then be used to easily to link to PubMed. This service is frequently used by publishers or other database providers who wish to link from bibliographic references on their web sites directly to entries in PubMed.

IV. ICP Mass Spectrometry

Inductively coupled plasma—mass spectrometry is an analytical technique which requires the sample to be introduced to a high temperature plasma, commonly argon, which dissociates molecules and ionizes atoms. The ions are passed into vacuum via a sample and skimmer cone interface, where a lens stack focuses the ion beam into a quadrupole mass spectrometer. Here, the ions are sorted by mass and detected using a scanning electron multiplier. Many models of ICP-MS are currently commercially available. Such as VG PlasmaQuad II ICP-MA by Fisons. A number of other vendors, such as PerkinElmer, LECO, ThermoQuest, etc. also manufacture a number of models of ICP-MS.

Some of the highlights of the ICP-MS technique are:

The detection limit for most elements is in the sub-parts per billion (ppb) range. For some elements it may lie in the sub parts per trillion range.

The versatility of the ICP-MS technique makes it a multi-disciplinary analytical tool.

Class 1000 clean room facilities ensure contamination-free sample preparation.

A number of different sample introduction techniques can be used with ICP-MS.

Electrothermal Vaporization (Graphite Furnace): The VG Mark IIIa Electrothermal Vaporization (ETV) Unit is a typical such sample introduction device. The ETV is most useful where sample sizes are small and quantification of trace to ultra-trace elements is required. High sensitivity is achieved through desolvating the sample prior to analysis as this reduces matrix and interference effects. The ETV has applicability in, inter alia, biological samples, as well as in the drug industry. ETV can also be used to track plutonium in the environment to the femtogram level.

Flo-Injection (for concentrated solutions): Flo-injector (such as the FISONS VGS 100 Flo-injector) allows a discrete sample volume to be injected into a continuously flowing carrier stream. Flow injection methodology has the following advantages over continuous nebulization where: i) sample pretreatment is necessary involving separation and pre-concentration; (ii) large dilution factors are required; (iii) there is limited sample volume; (iv) samples have a high dissolved solids content; (v) a range of calibration standards is required; (vi) standard additions are required; (vii) variations in solution properties may affect continuous nebulization.

Hydride generator (for hydrocarbon-rich samples): Hydride generator (such as the FISONS VGS 200 Hydride Generator) is a specialized sample introduction apparatus which allows enhanced detection limits from those elements that form gaseous hydrides at ambient temperatures (i.e., As, Bi, Ge, Pb, Sb, Se, Sn, Te). For example,

NaBH₄+3H₂O+HCl═H₃BO₄+NaCl+8H+X═EHn+H₂

where X is the element of interest. This apparatus may also be used to generate mercury vapor. This can be used for water and biological samples.

Autosampler (for large sample batches): Autosample, such as the Gilson 222 Autosampler, is generally used for high sample throughput situations. For example, the Gilson 222 autosampler has four racks of 44 samples/standards/blanks can be set up with the fifth rack being used for differential washing (3 washes) between individual analyses in order to prevent cross contamination. A three-wash sequence (10% HNO₃ with one drop of HF per 100 ml, 10% HNO₃, and 5% HNO₃) minimizes memory effects especially over extended runs. Other commercially available autosamplers or user-improved models may also be used with the instant invention.

UltraSonic Nebulizer (for ultratrace element analyses in the parts per quatrillion-ppq-range): CETAC 5000 Ultrasonic Nebulizer is a sample introduction apparatus that bypasses the spray chamber. The liquid sample is introduced to a transducer plate which creates the mist. This mist is taken via an argon flow along a tube where it is desolvated by heating and cooling in rapid succession, on its way to the plasma. This creates a higher signal to background ratio, thus increasing sensitivity. Using the Ultrasonic Nebulizer increases sensitivity by an order of magnitude, on average. Detection limits can be lowered to sub-part-per-trillion levels.

Laser sampling system (for solid samples): The LaserProbe (such as the VG LaserProbe) offers solid sampling capabilities with good spatial resolution and reduces and/or eliminates oxide/nitride/chloride/hydride interferences through the analysis of a dry sample. The laser beam of the VG LaserProbe is typically ˜20-25 μm in diameter at a wavelength of 1064 nm in the infra-red range. The LaserProbe can be used in laser ICP-MS to analyze trace element contents of a sample, such as a thin biological section. The ideal situation is that we can take a thin section from the Electron Microprobe from which major and minor element data have been obtained. These data can then be used as internal standards for the trace element analysis on the LaserProbe. The LaserProbe can be upgraded to include laser radiation in the visible (532 nm) and ultra-violet (266 nm) ranges.

The use of a laser in ICP-MS has allowed the geochemical analysis of small, solid samples to be accomplished. In order to give an insight to the potential of LA-ICP-MS.

Laser ablation ICP-MS (LA-ICP-MS) is incredibly versatile. In theory, any solid material can be analyzed provided the laser can couple with the material, external standards are available, and internal standards are known. The advantages of LA-ICP-MS over conventional solution nebulization ICP-MS have been reported by many authors (e.g., Denoyer et al., 1991, Anal. Chem., 63, 445A-457A; Jarvis and Williams, 1993, Chem. Geol., 106, 251-262; and Longerich et al., 1993, Geoscience Canada, 20, 21-27): (A) Analysis of solid samples is direct and requires no lengthy dissolution processing which may be incomplete and can also potentially introduce contamination to the sample; (B) Analysis of solid samples by LA-ICP-MS requires little preparation (a flat surface may be required if the entire sample is to be probed, but it need not be parallel to better than 200 μm provided that the focus of the laser does not change from one part of the sample to another, resulting in different ablation characteristics); (C) a dry sample is introduced to the plasma with a resulting lack of polyatomic interference species produced by the interaction of water and acid species with the argon plasma.

Compared to other microsampling analytical techniques, LA-ICP-MS has several distinct advantages: 1) Laser probing utilizes light rather than charged particles and can, therefore, analyze both conducting and non-conducting material without the need for a conductive coat and/or other charge balancing techniques, as in SIMS and electron microprobe techniques; 2) no vacuum is required in the sample chamber, although an airtight seal is; 3) LA-ICP-MS, unlike Atomic Emission Spectroscopy, separates the ionization step from the sampling step—the laser is used to ablate the sample only and the material is transported to the secondary plasma source in the torch of the ICP. Therefore, both steps can be independently controlled and optimized; 4) the high sensitivity of the ICP-MS allows small samples to be quantified, which is ideal for LA-ICP-MS in that spatial resolution can be used to investigate compositional gradients across a sample, even though the laser sampling area is 5-10 times greater than that obtained for the electron or ion microprobes (Reed, 1989, Mineral. Mag. 53, 3-24; and Reed, 1990, Chem. Geol., 83, 1-9). However, the spatial resolution and detection limit of LA-ICP-MS is being constantly reduced for in situ analysis of solid samples (e.g., Jackson et al., 1992, Canadian Mineral., 30, 1049-1064; Pearce et al., 1992a, J. Anal. Atom. Spectrom., 7, 53-57; Neal, 1993, Eos Trans. AGU, 74; Feng, 1994, Geochim. Cosmochim. Acta, 58, 1615-1623). For example, Gray (Analyst, 110, 551-556, 1985) reported a pit diameter of 700 μm, whereas Jackson et al. (Canadian Mineral., 30, 1049-1064, 1992) and Neal (Eos Trans. AGU, 74, 626, 1993) reported pit diameters of 20-30 μm—a 96% decrease over 7-8 years. Finally, trace-element analysis using LA-ICP-MS does not require involved interference corrections inherent in SIMS analysis and the hardware is considerably cheaper. Given this proviso, it has been found that a larger number of elements can be accurately quantified by LA-ICP-MS over SIMS, provided well characterized standards are available, with a detection limit similar to that of SIMS (Denoyer et al., 1991, Anal. Chem., 63, 445A-457A).

The laser light emitted using a Nd:YAG laser is generally at 1064 nm in the infra-red range. This wavelength couples easily with samples containing significant quantities of the transition elements. Longerich et al. (Geoscience Canada, 20, 21-27, 1993) incorporated a harmonic generator into the laser apparatus which allowed shorter wavelength (532 nm and 266 nm) laser radiation to be generated. Jenner et al. (Geochim. Cosmochim. Acta, 58, 5099-5103, 1994) determined crystal-matrix partition coefficients for a variety of trace elements using 266 nm wavelength laser radiation and reported a fourfold decrease in the diameter of the ablation pit from that produced at 1064 nm on this particular LA-ICP-MS system. This is important for controlled ablation of transition-element-poor materials (e.g., the minerals calcite and feldspar). However, Abell (In Applications of Plasma Source Mass Spectrometry, edited by G. Holland and A. N. Eaton, pp. 209-217. The Royal Society of Chemistry, 1990) noted that materials which are transparent to laser light could be ablated using the 1064 nm wavelength if the laser pulse has sufficient energy. Feng (Supra, 1994) used this modus operandi to undertake controlled ablation and analysis of carbonates using 1064 nm laser radiation.

The laser may be operated in two modes: (a) “Q-Switched’” where a short laser pulse (10 ns) contains practically all of the energy; and (b) “Fixed-Q'” or “Free-Running” where the laser pulse is much longer (120-150 sec) and the power delivered is considerably less (see Denoyer et al., supra, 1991, for detailed descriptions). The resulting ablation characteristics are very different and produce very different ablation pits, thus affecting the size of the sample analyzed. In Q-switched mode, the laser energy is higher (relative to the free-running mode), and much of the ablation occurs through total vaporization and mechanical ablation. Calculated Relative Sensitivity Factors (RSFs) are relatively uniform across the mass range (e.g., Denoyer et al., supra, 1991). In Fixed-Q or Free-Running mode, the power of the laser is lower, the laser interacts with the sample for a longer period of time and is conducted more deeply into the sample. This produces a deeper crater of smaller diameter relative to Q-switched mode, but the elements are ablated selectively on the basis of their vaporization energies (e.g., Thompson et al., 1990, J. Anal. Atom. Spectrom., 5, 49-55). This fractionation produces variable RSFs across the mass range relative to those produced in Q-switched mode. Generally, the laser is operated in Q-switched mode.

By its very nature, the signal induced by the laser pulse is a transient one, thus making tuning difficult even in Q-switched mode. Hollocher (Rev. Sci. Instrum., 64, 2395-2396, 1993) reported a technique involving the by-pass of the argon carrier from the sample chamber over a crystal of iodine held in a glass tube. Iodine is evaporated at room temperature, is monoisotopic having an atomic weight of 127 which is in the middle of the mass range, and is relatively resistant to forming polyatomic species (i.e., ArI). While the memory of iodine may be long in the system, if this element does not need to be quantified and is only used for tuning, such a set up would seem ideal for LA-ICP-MS.

Detection limits are intimately related to the signal intensity, counting time per element for the ablation mass, and on the sample cell design which affects the size and configuration of the ablation pit and, thus, on the amount of material ablated. The precision of LA-ICP-MS is dependent on signal fluctuations as a result of pulse-to-pulse variations in the amount ablated and hence the amount reaching the plasma (van de Weijer et al., 1992, J. Anal. Atom. Spectrom., 7, 599-603). A quantitative analysis of both major and trace elements in geological samples can be obtained by normalizing the intensities of the observed peaks to either the weight of the sample removed or a true internal standard [e.g., Imai, 1990, Anal. Chim. Acta, 235, 381-391; Denoyer et al., 1991, supra). Determining the accurate weight of sample removed is an extremely involved process, especially as not all of the material ablated reaches the plasma or collector (e.g., Remond et al., 1990, Scanning Microscopy, 4, 249-274). Internal standardization removes the need of knowing an accurate volume of material ablated and amount transported to the ICP torch. Also, normalizing signals from the unknown sample to an internal standard concentration removes any change in response with time between analyses (e.g., Pearce et al., 1992a, J. Anal. Atom. Spectrom., 7, 53-57; Pearce et al., 1992b, J. Anal. Atom. Spectrom., 7, 595-598). However, this requires a knowledge of matrix composition and if it has an isotopic abundance which is less than 1% of the total matrix (van de Weijer et al., 1992, J. Anal. Atom. Spectrom., 7, 599-603). Choice of an internal standard is critical in that its behavior during ablation must be representative of the unknown elements being quantified (c.f., Jarvis and Williams, 1993, Chem. Geol., 106, 251-262). If knowledge of the matrix is known, then such data can be used as internal standards. This is of particular significance for geological applications, where major and minor elements are usually determined via other methods (i.e., electron microprobe for minerals and XRF or INA for bulk samples).

The requirement of careful matrix matching in order to obtain quantitative analyses of small samples via LA-ICP-MS is well documented in the recent literature (e.g., Denoyer et al., 1991, supra; Jarvis and Williams, 1993, Chem. Geol., 106, 251-262, 1993). In a study of pressed powder standard reference materials, Williams and Jarvis (1993) concluded that geological standards for LA-ICP-MS should not only be matched in chemistry, but more importantly in mineralogy. This is a particularly critical observation for the analysis of small geological samples which will tend to be individual minerals. However, it has been demonstrated that if the laser pulse has sufficient energy to ablate the sample via plasma plume expansion and not from absorption of the laser beam with resulting thermal vaporization (and matrix-dependent element fractionation), then nonmatrix matched standards may be used (e.g., Abell, 1990; Jackson et al., 1992; Jenner et al., 1994; Feng, 1994). Note that all procedures using nonmatrix matched standards are conducted in Q-switched mode which produces a more intense but shorter duration laser pulse (see above).

In an exemplary ICP-MS unit, an argon plasma can be used to volatilize (where applicable), atomize and ionize samples. For example, in the VG PlasmaQuad II ICP-MA, a magnetic field induced by an RF generator is placed at the end of the torch by the load coil. A “spark” of electrons from the tesla coil ignites the plasma by causing collisions between the electrons and Ar atoms induced by the magnetic field, resulting in creation of Ar⁺ and more electrons and so the process becomes self-sustaining. The temperature adjacent to the load coil is approximately 10,000 K, creating a lot of Ar⁺. Three Ar flows are introduced to the torch: 1) Cool Gas—the outer flow ˜14 l min⁻¹ keeps the sides of the torch from melting; 2) Auxilliary Flow—this is the intermediate flow through the torch that keeps the plasma away from the end of the torch at a rate of 0.5-1.5 l min⁻¹; and 3) Sample Flow—this central flow introduces the sample to the plasma at ˜0.7-1.0 l min⁻¹. The cool sample injected through the center of the plasma cools it to ˜7,000 K which reduces the abundance of Ar⁺ but still maximizes sample ionization.

The ICP-MS requires ultrapure water system to achieve its full potential. Ultrapure water is essential in the preparation of standards, the washing of glassware and cones, as well as being essentail for blank preparation. The ultrapure water system can be maintained by an incoming supply of softened water at 70μ which undergoes reverse osmosis followed by a final “polishing” to remove any impurities that still exist. A typical ultrapure water system can supply 5-8 liters of ultrapure water per hour. Other models of ultrapure water systems may also be used in the instant invention.

V. Exemplary Embodiments

In one aspect, the present invention provides a method for the evaluation of the phosphorous-related enzymatic activity of biological samples using ICP-MS. The specific embodiment described focuses on the activity of protein (native and recombinant) samples, however the method can also be adapted for use with other biological sample types, such as nucleotides, non-protein cellular components, cultured cells, biopsies, and tissues. The phosphorous-related activities that could be measured using this invention include, inter alia, kinase activity, phosphatase activity, and autophosphorylation. Furthermore, the effect of small molecules on these activities (e.g., inhibition or activation) can also be directly measured by adding the small molecules to the reaction solution and observing any variation on the P/S measurements.

To further illustrate, the subject method can be employed using samples arrayed in traditional 96 or 384 well plate formats. However, the flexibility of the assay protocols combined with the ability to automate to liquid transfer steps allows for any sample array format to be used. This could include arrays of test tubes, petri dishes, or vials. Furthermore, the samples could be analyzed from microfluidic arrays such as etched chips, beads, or fibers. Where laser ablation ICP-MS is used to detect phosphorous and sulfur levels, the samples can be arrayed on solid supports, including supports that can also be used for MALDI analysis (e.g., sequencing) of the samples.

The well plates (or similar array) can be coated with a wide variety of substrates to give great flexibility to the method. These include:

A kinase or phosphatase. The enzyme, either native or synthetic, can be directly attached to the well-plate surface by chemical means in order to evaluate its activity.

The test polypeptide. The potential substrate on which a kinase or phosphatase may (or may not) act upon can be chemically attached to the well-plate surface.

Antibodies. Antibodies with specific or non-specific binding characteristics can be attached to the well-plate surface so that proteins to be assayed can be isolated from solution.

The present method can also be used to generally determine the phosphorylation “state” of a sample of cells. Merely to illustrate, by culturing living cells or tissues on the well plate surface, fixing them (with methanol), and analyzing the lysate to determine the P/S levels, a broad measure of the total amount of phosphorylated proteins can be measured. In certain embodiments, only certain proteins may be isolated from the lysate for analysis, such as a set of proteins known to be regulated by phosphorylation and (optionally) being part of the same signalling pathway or having common features, such as being related enzymes, transcription factors, or the like. This allows for a basic determination of the effects of chemical stimulants on the phosphorylation pathways of the cultured cells or tissues.

The invention offers a number of significant advantages for the measurement of kinase and phosphatase activities, including:

High sensitivity. For instance, the use of ICP-MS offers unparallel sensitivity for measurements of phosphorous and sulfur atoms. Typically, the method allows chemical resolution of P⁺ and S⁺ at the sub-ppb (sub-femtogram/microliter) level.

Both P and S are measured simultaneously. The scanning ability of an ICP-MS machine or the like allows for concurrent measurement of these variables, eliminating the need for parallel experimental measurements.

Adaptability to automation. The use of well plates (or similar arrays of liquid volumes or dried spots) and relatively simple sample transfer protocols allow for the procedure to be automated using commercially available systems.

High speed. Coupled to a commercially available autosampler, the invention could achieve sample analysis rates faster than 1 minute per sample or less than 90 minutes for a 96-well plate.

Use in high-throughput screening. The high speed and automation capability of the invention allows for its use in high-throughput screening of kinase or phosphatase (or related) activities.

Moreover, the method of the present invention described also has a number of advantages compared to previously described methods to measure kinase activity. For instance, antibodies are not required. Previous methods to measure phosphorylation often require the use of antibodies which are often difficult to obtain and expensive. Furthermore, antibodies for phospho-serine and phospho-threonine are known to be very non-specific in their binding abilities. Fluorescent tracers are not required. Previous methods to measure kinase activity often rely on fluorescent measurements that are prone to high background and low sensitivity. Radioactive reagents are not required. Previous methods to measure kinase activity often rely on the use of radiolabeled compounds which have limitations due to their expense, health effects, and the need for careful handling methods.

As set forth above, the peptide samples for analysis by the present invention can be obtained, and supplied to the mass spectrometer, by various different standard methods. Desirably, the sample may be enriched for particular proteins using affinity chromatography or by immunoprecipitation using antibody to a particular polypeptide.

For further illustration, an example of the use of the subject method to assay for kinase activity can involve the following steps:

The protein(s) of interest are attached to the bottom of individual wells of a standard well plate. This can be accomplished by direct chemical attachment or biologically by affinity tagging.

Wells are washed with kinase buffer.

ATP is added to alternate wells to allowing any kinase reactions to proceed.

Samples from individual wells are prepared for analysis by ICP-MS and the P/S ratio is determined. Differences in the P/S ratio between samples with or without ATP added indicate the presence of kinase activity.

In certain embodiments, the subject method utilizes laser ablation ICP-MS. Analysis of solid samples by LA-ICP-MS requires little preparation (a flat surface may be required if the entire sample is to be probed, but it need not be parallel to better than 200 μm provided that the focus of the laser does not change from one part of the sample to another, resulting in different ablation characteristics); a dry sample is introduced to the plasma with a resulting lack of polyatomic interference species produced by the interaction of water and acid species with the argon plasma.

In preferred embodiments of the invention, the present method is applied to identify proteins which have been modified to include, or loss, phosphorylated amino acid residues such as phosphotyrosine, phosphoserine, phosphothreonine, phosphohistidine, phosphoarginine, phospholysine, phosphocysteine, phosphoglutamic acid and phosphoaspartic acid.

The following describes a specific example of a protocol for measurement of the autophosophorylation abilities of a protein domain EphA4:

A GST-EphA4 kinase domain fusion protein was prepared.

1. MaxiSorp 96-multiwell plates were coated with 1 mM glutathione prepared in TBS (Tris Buffered Saline, pH 7.5).

2. Wells washed with TBS.

3. Wells were incubated with various concentrations of GST-EphA4. This reaction ensures correct binding of the kinase to the well bottom.

4. Samples wells were washed with kinase buffer (20 mM HEPES, 5 mM Mg²⁺, 2 mM Mn²⁺).

5. Alternative well-plate rows were filled with 2 mM ATP.

6. The reaction was allowed to proceed at 37° C. for 1 hour.

7. Wells were stringently washed with TBS.

8. Samples were prepared for P/S analysis by addition of 50 μL HCL con. and 200 μL of water.

9. P/S ratios were determined using ICP-MS as described in references.

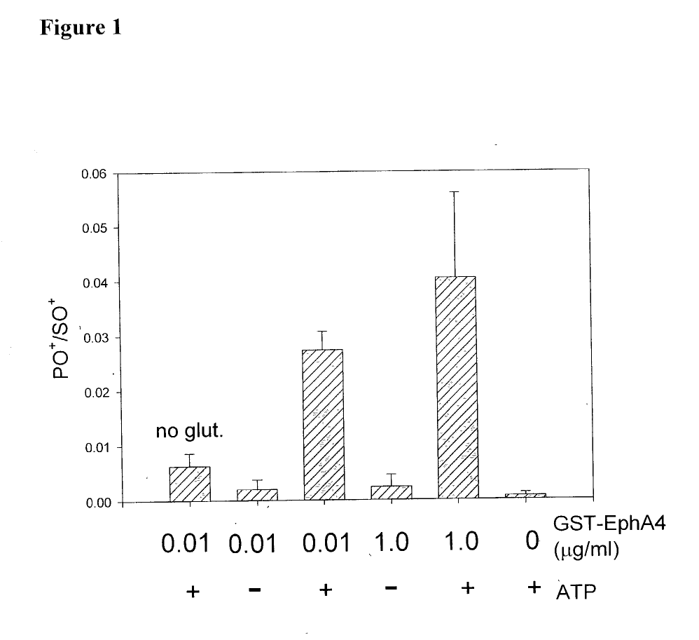

The results of this experiment are shown in the FIG. 1.

A similar experiment was conducted to measure the kinase activity of synthetic kinase substrate:

1. MaxiSorp plates were coated with 20 μg/ml poly(Glu, Tyr), a synthetic kinase substrate.

2. Solution containing GST-EphA4 kinase domain at various concentrations in kinase buffer both with ATP (+ATP) or without ATP (−ATP).

3. The well plate was incubated at 37° C. for 1 hr.

4. Samples were prepared for analyzed for PO⁺/SO⁺ as described above.

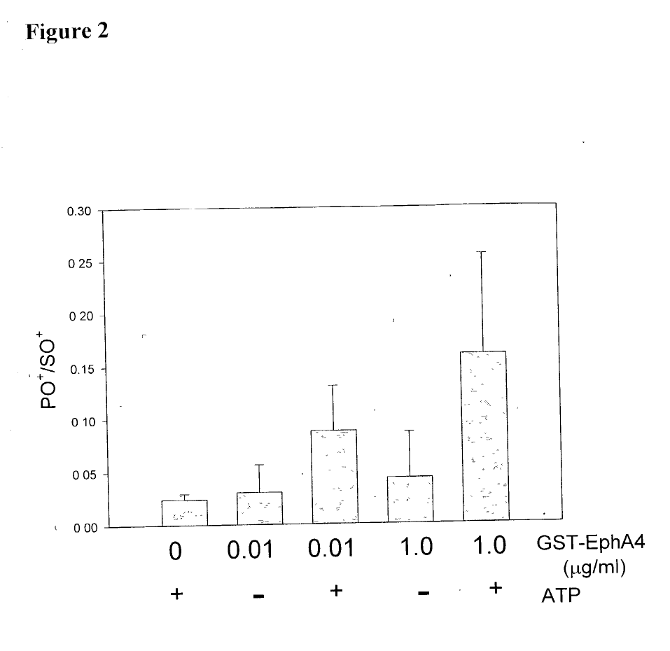

Results are shown in the FIG. 2.

In still other embodiments, the subject method can be used to determine changes in sulfation of test polypeptide, or the sulfation state of a cell. Sulfate modification of proteins occurs at tyrosine residues such as in fibrinogen and in some secreted proteins (e.g., gastrin). A modulator of extracellular protein-protein interactions—tyrosine sulfation is a post-translational modification of many secreted and membrane-bound proteins. Recent work has implicated tyrosine sulfate as a determinant of protein-protein interactions involved in leukocyte adhesion, hemostasis and chemokine signaling.

Work during the past 10 years has established that tyrosine sulfation is a posttranslational modification that occurs in essentially all eukaryotic cells containing a Golgi apparatus. As compared to other various posttranslational covalent modifications of proteins, O-sulfation on tyrosine residues has until recently attracted relatively little attention because it was considered a rare modification. The presence of a sulfated tyrosine residue was first detected in fibrinopeptide B, then on gastrin and CCK1, and was more recently shown to occur in a rather large number of secretory proteins such as immunoglobulin G, fibronectin, and procollagens. For many proteins, tyrosine sulfation appears to be important for biological activity and correct cellular processing. The loss of sulfated tyrosine residues decreases the interactions between factor VIII and von Willebrand factor, hirudin and thrombin, fibronectin and fibrin, complement C4 and C1s, and leuserpin 2 and thrombin. Studies with P-selectin glycoprotein ligand (PSGL) have shown that a sulfated peptide segment of the amino terminus of PSGL-1 is critical for P-selectin binding. Tyrosine sulfation of chemokine receptor CCR5 facilitates HIV-1 entry. The proinflammatory cytokine tumor necrosis factor was found to convert CD44 from its inactive, nonbinding form to its active form by inducing the sulfation of CD44. Sulfation was thus shown as a potential means of regulating CD44-mediated leukocyte adhesion at inflammatory sites. Correlative studies on the degree of gastrin sulfation and its processing suggest that sulfated gastrin 34 is more readily processed to gastrin 17. Mutational analysis of tyrosine sulfation of gastrin demonstrated that substitution of the alanyl residue N-terminal to the sulfated tyrosine with an acidic residue promotes sulfation and complete sulfation increases the endoproteolytic processing of progastrin. On the basis of this observation, it was also suggested that tyrosine sulfation is an important regulator of phenotypic gene expression.

Two members of sulfotransferases responsible for peptide sulfation localized in the trans-Golgi network were recently cloned, tyrosylprotein sulfotransferase TPST-1 and TPST-2.

In addition to uses similar to that described for assessing the phosphorylation status of individual polypeptides and cells, the subject method can also be used to assess changes in the sulfation status of proteins found in bodily fluids, such as serum, urine, cerebral spinal fluid, lymph, etc.

The method can also be extended to broadly determine the phosphorylation “state” of the cells. By culturing living cells or tissues on the well plate surface, fixing them (with methanol), and analyzing the lysate to determine the P/S levels, a broad measure of the total amount of phosphorylated proteins can be measured. This allows for a basic determination of the effects of chemical stimulants on the phosphorylation pathways of the cultured cells or tissues.

The following example demonstrats that very small amount of biopsy material can be used to distinguish normal from malignant tissue in human patient.

To illustrate, fine-needle aspiration biopsy material can be frozen-crushed to powder and dissolved in HCl for further phosphate determination according to the following protocol.

1. Liquid nitrogen snap-frozen tissue samples are ground into fine powder with a liquid nitrogen-cooled mortar and pestle.

2. Approximately 1-5 mg of tissue powder is weighed out on an analytical scale.

3. Tissue powder is lysed/digested in 1 ml conc. HCl (37% high purity grade).

4. Samples are diluted with ddH₂O (1:100) and analysed by ICP/MS. Values are acquired for PO and SO. The normalized ratio PO/SO is used as a read out.

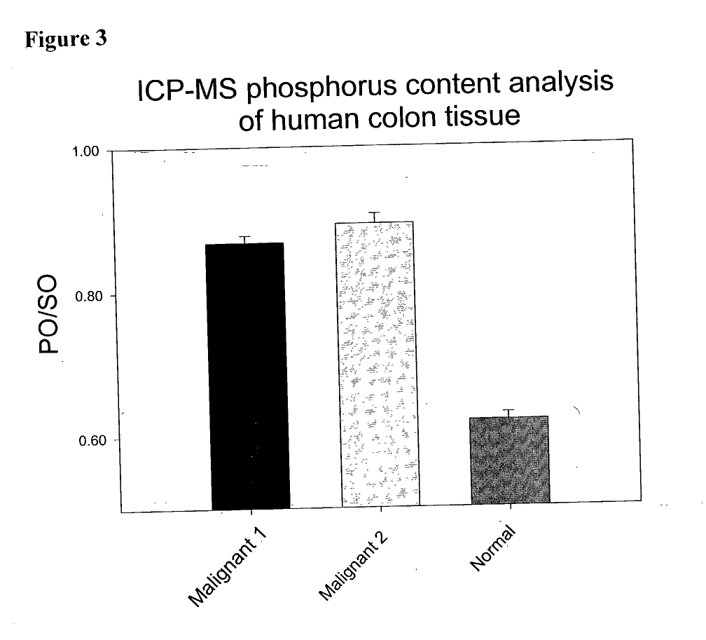

Results for PO/SO ratio difference between human normal colorectal epithelium and human colorectal carcinoma sample are shown in FIG. 3. Both samples were obtained from the same patient. Amount of material used is extremely low—1 mg, and only 1% was used for the ICP-MS analysis. Thus, very small amount of biopsy material can be used to distinguish normal from malignant tissue. In addition, as shown in this example, technically, it is very easy and routine to obtain human tissue samples through, for example, biopsy. The instant invention thus provides a diagnosis method to differentiate normal from disease tissues based on their differences in P and S content ratio.

We claim:

1. A method for identifying the phosphorylation state of a polypeptide, comprising:
(i) determining, by mass spectroscopy, an elemental ratio of phosphorous to sulfur in a test sample of a polypeptide prepared under test conditions, and
(ii) comparing the ratio of phosphorous to sulfur for the test sample with a ratio of phosphorous to sulfur for one or more reference samples of the test polypeptide, the reference samples being prepared under defined phosphorylation conditions,
wherein a difference in the ratio of phosphorous to sulfur between the test and reference polypeptide samples indicates a difference in the level of phosphorylation resulting from the test conditions.

2. A method for identifying the sulfation state of a polypeptide, comprising:
(i) determining, by mass spectroscopy, an elemental ratio of phosphorous to sulfur in a test sample of a polypeptide prepared under test conditions, and
(ii) comparing the ratio of phosphorous to sulfur for the test sample with a ratio of phosphorous to sulfur for one or more reference samples of the test polypeptide, the reference samples being prepared under defined sulfation conditions,
wherein a difference in the ratio of phosphorous to sulfur between the test and the reference polypeptide samples indicates a difference in the level of sulfation resulting from the test conditions.

3. The method of claim 1 or 2, further comprising determining at least a portion of the sequence of a polypeptide identified by a difference in the level of phosphorylation or sulfation between the test and the reference polypeptide samples.

4. The method of claim 3, further comprising searching one or more sequence databases for polypeptides, or the coding sequences therefor, having identical or homologous sequences to that determined for the identified polypeptide.

5. The method of claim 1, wherein the test conditions include exposing the test polypeptide to a kinase under conditions wherein phosphorylation of the test polypeptide occurs if it is a substrate of the kinase.

6. The method of claim 1, wherein the test conditions include exposing a phosphorylated form of the test polypeptide to a phosphatase under conditions wherein dephosphorylation of the test polypeptide occurs-if it is a substrate of the phosphatase.

7. The method of claim 2, wherein the test conditions include exposing the test polypeptide to a tyrosylprotein sulfotransferase under conditions wherein sulfation of the test polypeptide occurs if it is a substrate of the sulfotransferase.

8. The method of claim I or 2, wherein the method is carried out on a library of different test polypeptides.

9. The method of any of claims 1-8, wherein the test conditions and/or the defined conditions include a whole cell in which the test polypeptide is expressed.

10. The method of any of claims 1-8, wherein the test conditions and/or the defined conditions include a cell lysate or purified protein composition.

11. The method of claim 1, 2, 8, 9 or 10, wherein the test polypeptide is separated from other polypeptides present in the test conditions using one or more of liquid chromatography, gel-filtration, isoelectric precipitation, electrophoresis, isoelectric focusing, ion exchange chromatography, and affinity chromatography.

12. The method of claim 11, wherein said polypeptides are separated using high performance liquid chromatography.

13. The method of claim 1, 2, 8, 9, or 10, wherein the test polypeptide is separated from other polypeptides present in the test conditions on the basis of size, solubility, electric charge, and/or ligand specificity.

14. The method of any of claims 1-13, wherein the mass spectroscopy step uses inductively coupled plasma mass spectrometry (ICP-MS).

15. The method of claim 14, wherein the mass spectroscopy step uses laser ablation ICP-MS.

16. The method of claim 3, wherein the sequence of the test polypeptide is determined from spectra obtained using a mass spectrometer in which ionization of the sample protein is accomplished by matrix-assisted laser desorption (MALDI) ionization, electrospray (ESI), or electron impact (EI).

17. A method for identifying a substrate for a kinase, comprising:
(i) contacting a test sample of a polypeptide with a kinase under conditions wherein phosphorylation of the test polypeptide occurs if it is a substrate of the kinase,
(ii) determining, by mass spectroscopy, an elemental ratio of phosphorous to sulfur in the test sample, and
(iii) comparing the ratio of phosphorous to sulfur for the test sample with a ratio of phosphorous to sulfur for a reference sample of the test polypeptide not treated with the kinase,
wherein an increase in the ratio of phosphorous to sulfur between the test and reference samples indicates that the test polypeptide is a substrate for the kinase.

18. A method for identifying a substrate for a phosphatase, comprising:
(i) contacting a phosphorylated sample of a test polypeptide with a phosphatase under conditions wherein dephosphorylation of the test polypeptide occurs if it is a substrate of the phosphatase,
(ii) determining, by mass spectroscopy, an elemental ratio of phosphorous to sulfur in the test sample, and
(iii) comparing the ratio of phosphorous to sulfur for the phosphorylated sample with a ratio of phosphorous to sulfur for a reference sample of the test polypeptide not treated with the phosphatase,
wherein a decrease in the ratio of phosphorous to sulfur between the test sample and reference sample indicates that the phosphorylated test polypeptide is a substrate for the phosphatase.

19. A mass spectrometry system including a module that identifies the phosphorylation state of a test peptide, which module determines a level of elemental phosphorous and a level of elemental sulfur in a test sample of a polypeptide, and calculates an elemental ratio of phosphorous to sulfur for the test sample.

20. A method of conducting a drug discovery business, comprising:
(i) by the method of any of claims 1-19, identifying a kinase or phosphatase and substrate thereof;
(ii) identifying agents by their ability to alter a level of phosphorylation of the substrate;
(iii) conducting therapeutic profiling of agents identified in step (ii), or further analogs thereof, for efficacy and toxicity in animals; and
(iv) formulating a pharmaceutical preparation including one or more agents identified in step (iii) as having an acceptable therapeutic profile.

21. A method of conducting a drug discovery business, comprising:
(i) by the method of any of claims 1-19, identifying substrate proteins which are phosphorylated or dephosphorylated as compared between two different states of a cell;
(ii) identifying agents by their ability to alter a level of phosphorylation of the substrate protein(s);
(iii) conducting therapeutic profiling of agents identified in step (ii), or further analogs thereof, for efficacy and toxicity in animals; and
(iv) formulating a pharmaceutical preparation including one or more agents identified in step (iii) as having an acceptable therapeutic profile.

22. The method of claim 21, wherein the two different states compared are normal and diseased states, or differentiated and undifferentiated, or resting and activating, or induced and uninduced.

23. The method of claim 20, including an additional step of establishing a distribution system for distributing the pharmaceutical preparation for sale, and, optionally, establishing a sales group for marketing the pharmaceutical preparation.

24. A method of conducting a proteomics business, comprising:
(i) by the method of any of claims 1-19, identifying a kinase or phosphatase and substrate thereof;
(ii) licensing, to a third party, rights for further drug development of agents that alter a level of phosphorylation of the substrate.

25. A method for determining the phosphorylation state of a cell, comprising:
(i) determining, by mass spectroscopy, an elemental ratio of phosphorous to sulfur in a test sample of polypeptides prepared from one or more cells of a first phenotype, and
(ii) comparing the ratio of phosphorous to sulfur for the test sample with a ratio of phosphorous to sulfur for one or more reference samples of the polypeptides, the reference samples being prepared from one or more cells of a second phenotype,
wherein a difference in the ratio of phosphorous to sulfur between the test sample and the reference sample indicates a difference in a level of phosphorylation between the first and second phenotypes.

26. A method for determining the kinase activity of a kinase, comprising:
(i) contacting a test sample of a polypeptide with a kinase under conditions wherein phosphorylation of the test polypeptide occurs,
(ii) determining, by mass spectroscopy, a first elemental ratio of phosphorous to sulfur in the test sample at a first time, and
(iii) determining, by mass spectroscopy, a second elemental ratio of phosphorous to sulfur in the test sample at a second time,
whereby a difference between the first elemental ratio and the second elemental ratio and a difference between the first time and the second time are indicative of a rate constant for the kinase.

27. A method for determining the phosphatase activity of a phosphatase, comprising:
(i) contacting a test sample of a phosphorylated polypeptide with a phosphatase under conditions wherein dephosphorylation of the polypeptide occurs,
(ii) determining, by mass spectroscopy, a first elemental ratio of phosphorous to sulfur in the test sample at a first time, and
(iii) determining, by mass spectroscopy, a second elemental ratio of phosphorous to sulfur in the test sample at a second time,
whereby a difference between the first elemental ratio and the second elemental ratio and a difference between the first time and the second time are indicative of a rate constant for the phosphatase.

28. A method for identifying the kinase activity of a polypeptide, comprising:
(i) contacting a test sample of a substrate with a test polypeptide under conditions wherein phosphorylation of the substrate occurs if the polypeptide has a kinase activity for the substrate,
(ii) determining, by mass spectroscopy, an elemental ratio of phosphorous to sulfur in the test sample, and
(iii) comparing the ratio of phosphorous to sulfur for the test sample with a ratio of phosphorous to sulfur for a reference sample of the substrate not treated with the test polypeptide,
wherein an increase in the ratio of phosphorous to sulfur between the test sample and the reference sample indicates that the test polypeptide has a kinase activity.

29. A method for identifying the phosphatase activity of a polypeptide, comprising:
(i) contacting a test sample of a phosphorylated substrate with a test polypeptide under conditions wherein dephosphorylation of the substrate occurs if the polypeptide has a phosphatase activity for the substrate,
(ii) determining, by mass spectroscopy, an elemental ratio of phosphorous to sulfur in the test sample, and
(iii) comparing the ratio of phosphorous to sulfur for the test sample with a ratio of phosphorous to sulfur for a reference sample of the substrate not treated with the phosphatase,
wherein a decrease in the ratio of phosphorous to sulfur between the test sample and reference sample indicates a phosphatase activity for the test polypeptide.

30. The method of claim 28 or 29, wherein the test polypeptide is a variant of a polypeptide that has a phosphatase or kinase activity for the substrate.

31. The method of claim 30, wherein the variant is a mutated or truncated variant of a polypeptide that has a phosphatase or kinase activity for the substrate.

32. A method for identifying an inhibitor of the kinase activity of a kinase, comprising:
(i) contacting a test sample of a polypeptide with a kinase and a test compound under conditions wherein phosphorylation of the polypeptide occurs in the absence of the test compound,
(ii) determining, by mass spectroscopy, an elemental ratio of phosphorous to sulfur in the sample, and
(iii) comparing the ratio of phosphorous to sulfur for the test sample with a ratio of phosphorous to sulfur for a reference sample of the polypeptide treated with the kinase in the absence of the test compound,
wherein a decreased ratio of phosphorous to sulfur in the test sample as compared to the reference sample indicates that the test compound inhibits the kinase activity.

33. A method for identifying an inhibitor of the phosphatase activity of a phosphatase, comprising:
(i) contacting a test sample of a phosphorylated polypeptide with a phosphatase and a test compound under conditions wherein dephosphorylation of test polypeptide occurs in the absence of the test compound,
(ii) determining, by mass spectroscopy, an elemental ratio of phosphorous to sulfur in the test sample, and
(iii) comparing the ratio of phosphorous to sulfur for the test sample with a ratio of phosphorous to sulfur for a reference sample of the substrate treated with the phosphatase in the absence of the test compound,
wherein an increased ratio of phosphorous to sulfur in the test sample as compared to the reference sample indicates inhibition of the phosphatase activity by the test compound.

33. A method for identifying an agonist of the kinase activity of a kinase, comprising:
(i) contacting a test sample of a polypeptide with a kinase and a test compound under conditions wherein phosphorylation of the polypeptide occurs in the absence of the test compound,
(ii) determining, by mass spectroscopy, an elemental ratio of phosphorous to sulfur in the sample, and
(iii) comparing the ratio of phosphorous to sulfur for the test sample with a ratio of phosphorous to sulfur for a reference sample of the polypeptide treated with the kinase in the absence of the test compound,
wherein an increased ratio of phosphorous to sulfur in the test sample as compared to the reference sample indicates that the test compound agonizes the kinase activity.

34. A method for identifying an agonist of the phosphatase activity of a phosphatase, comprising:
(i) contacting a test sample of a phosphorylated polypeptide with a phosphatase and a test compound under conditions wherein dephosphorylation of test polypeptide occurs in the absence of the test compound,
ii) determining, by mass spectroscopy, an elemental ratio of phosphorous to sulfur in the test sample, and
iii) comparing the ratio of phosphorous to sulfur for the test sample with a ratio of phosphorous to sulfur for a reference sample of the substrate treated with the phosphatase in the absence of the-test-compound,
wherein a decreased ratio of phosphorous to sulfur in the test sample as compared to the reference sample indicates that the test compound agonizes the phosphatase activity.
